(19) United States
(12) Patent Application Publication
(10) Pub. No.: US 2010/0167405 A1
(43) Pub. Date: Jul. 1, 2010

(54) PROCESSES FOR IMPROVED STRAIN ENGINEERING

(75) Inventor: James A. Williams, Lincoln, NE (US)

Correspondence Address: BROOKS KUSHMAN P.C., 1000 TOWN CENTER, TWENTY-SECOND FLOOR, SOUTHFIELD, MI, 48075, US

(73) Assignee: NATURE TECHNOLOGY CORPORATION, Lincoln, NE (US)

(21) Appl. No.: 12/601,505

(22) PCT Filed: May 22, 2008
(86) PCT No.: PCT/US08/06548

§ 371 Date: Nov. 23, 2009

Related U.S. Application Data
(60) Provisional application No. 60/931,890, filed on May 24, 2007.

(51) Int. Cl. C12N 15/70; C12N 15/00

(52) U.S. Cl. 435/476; 435/471; 435/440

(57) ABSTRACT

Improvements in strain engineering technology are needed to insure the economic feasibility of future engineered recombinant organisms for industrial biotechnology. Disclosed herein are rapid, efficient methods (Genome Mass Transfer) that facilitate introduction of new selectable traits into a target microbial host. In one preferred embodiment, methods for high efficiency electroporation mediated transfer of donor DNA into a recipient microbial cell are disclosed.

This application claims the benefit of Provisional Patent Application U.S. 60/931,890 filed 24 May 2007

### STATEMENT REGARDING FEDERALLY SPONSORED RESEARCH OR DEVELOPMENT

This invention was made in part with government support under Grant No. 2 R44 GM072141-02, awarded by the National Institutes of Health. The government has certain rights in this invention.

### FIELD OF THE INVENTION

The present invention relates to the engineering of bacterial or eukaryotic strains for academic or industrial applications.

### BACKGROUND OF THE INVENTION

The present invention relates to engineering novel bacterial or eukaryotic strains. Such strains are useful in biotechnology, transgenic organisms, gene therapy, therapeutic vaccination, agriculture and DNA vaccines.

With the invention in mind, a search of the prior art was conducted. Strain engineering, like cloning, is a fundamental technology utilized in biotechnology. Strain engineering is utilized to confer new traits onto existing strains.

The basic methods for strain engineering are well-known. One form of strain engineering involves transfer of a trait from one organism to another organism, of the same, or different species. Ideally, the transferred trait is selectable, by acquisition of a new phenotype in the recipient strain.

To transfer a trait from one organism to another, genomic DNA containing the trait may be transferred to the recipient. This may be accomplished for transfer of traits between E. coli strains using P1 transduction. This is a tedious and slow process.

Methods for electroporation of genomic DNA molecules into microbial cells (such as E. coli, yeast or agrobacterium) are known in the art (Charles T C, Doty S L, Nester E W. 1994. Appl. Environ. Microbiol. 60: 4192; Adam, A C, Gonzalez-Blasco G, Rubio-Texeira M, Polaina J. 1999. Appl. Environ. Microbiol. 65: 5303; Choi K H, Kumar A, Schweizer H P. 2006. J Microbiol. Methods 64: 391). Choi et al, Supra, 2006 teach the use of electroporation of genomic DNA to transfer traits between Pseudomonas strains, and

Charles et al, Supra 1994 teach to use electroporation of genomic DNA to, with very low efficiency, transfer traits between Agrobacterium strains. Adam et al, Supra, 1999, teach that transfer of genomic DNA from yeast to E. coli is inefficient and rare. In general, electroporation transfer using gDNA is inefficient, and the frequency of transfer too low for application in strain engineering in most organisms. Consistent with this, P1 transduction is still exclusively utilized for transfer of traits between E. coli strains.

### Current Barriers

Current strain engineering methods are not optimal for efficient transfer of traits between organisms.

Even in view of the prior art, there remains a need for an effective method to engineer strains.

### DISCLOSURE OF THE INVENTION

The invention is a method for trait transfer. In a preferred embodiment, a trait is transferred by recombination between isolated DNA and the genome of a recipient bacterium. In a preferred embodiment, a trait is transferred by recombination between isolated DNA and the genome of a recipient bacterium that has been rendered recombinogenic by expression of recombineering proteins. In another preferred embodiment, the DNA is not isolated from the donor organism prior to introduction to the recipient. In another preferred embodiment, the DNA is introduced into the recipient by electroporation. In another preferred embodiment, electroporation efficiency is enhanced by cationic lipids. In another preferred embodiment, the isolated DNA is genomic DNA. In another preferred embodiment, the isolated DNA is amplified prior to introduction to a recipient organism. In another preferred embodiment, the donor DNA is amplified directly from cell lysates prior to introduction to a recipient organism. In another preferred embodiment, the DNA amplification is performed using Phi29 DNA polymerase. In another preferred embodiment, electroporation of isolated or amplified genomic DNA is utilized to transfer traits between strains of E. coli. These processes dramatically improve the efficiency of gene transfer relative to the processes described in the art.

### BRIEF SUMMARY OF THE INVENTION

It is a purpose and/or objective of the present invention to provide processes to transfer traits between organisms. It is another purpose and/or objective of the present invention to provide processes to rapidly transfer traits between E. coli strains. Another disclosure is improved trait transfer methods that, compared to methods defined in the art such as P1 transduction, are improved by; simplified methodology by elimination of several complex steps and specialized bacteriophage transduction methods; increased speed due to elimination of multiple steps required for phage transduction; and adaptability to high throughput gene transfer.

Further objects and advantages of the invention will become apparent from a consideration of the drawings and ensuing description.

### BRIEF DESCRIPTION OF THE DRAWING FIGURES

FIG. 1. Genome Mass Transfer

FIG. 2. pKD46-RecApa plasmid, orientation 2

FIG. 3: Homologous recombination involving DNA strand invasion

FIG. 4. Red gam mediated gene replacement

Table 1: Strain engineering by Genome Mass Transfer

Table 2: RecA and lambda Red Gam requirements for GMT

Table 3: GMT of pAH144-C1857-tetR+

Table 4: GMT (Xja araB insert) versus PCR mediated gene knockout (mioC)

Table 5: GMT (mutS) versus PCR mediated gene knockout (spoT)

Table 6: GMT variability between loci

### DETAILED DESCRIPTION OF THE INVENTION

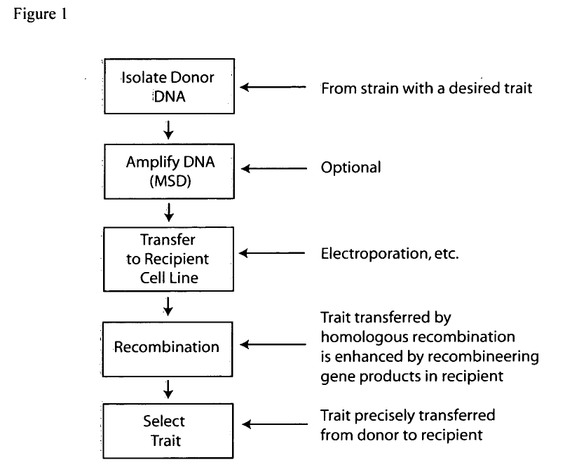

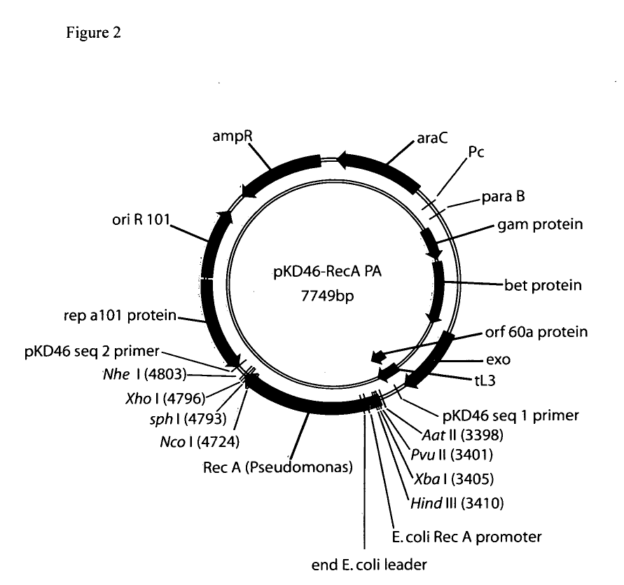

Turning now to the drawings, FIG. 1. shows use of ‘genome mass transfer’, optionally using amplified DNA as donor, to transfer a trait from one organism to another FIG. 2., shows pKD46-RecApa plasmid, orientation 2. Orientation 1 has the RecA (Pseudomonas) gene in the opposite orientation. The parent pKD46 plasmid does not have the RecA insert

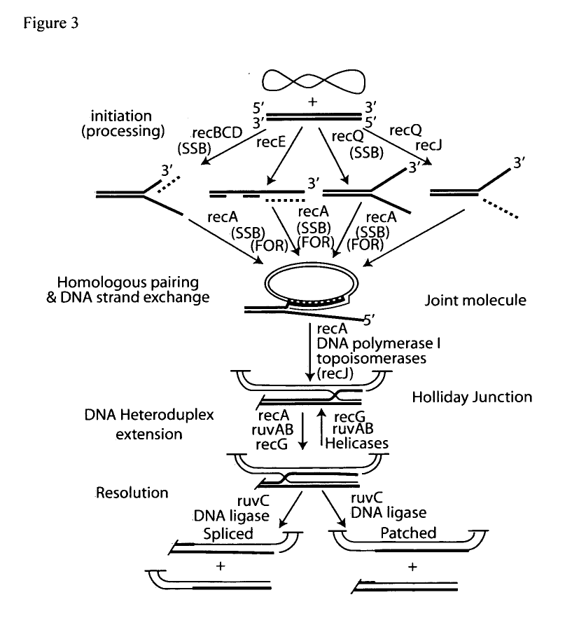

FIG. 3., summarizes the mechanisms of homologous recombination involving DNA strand invasion in E. coli

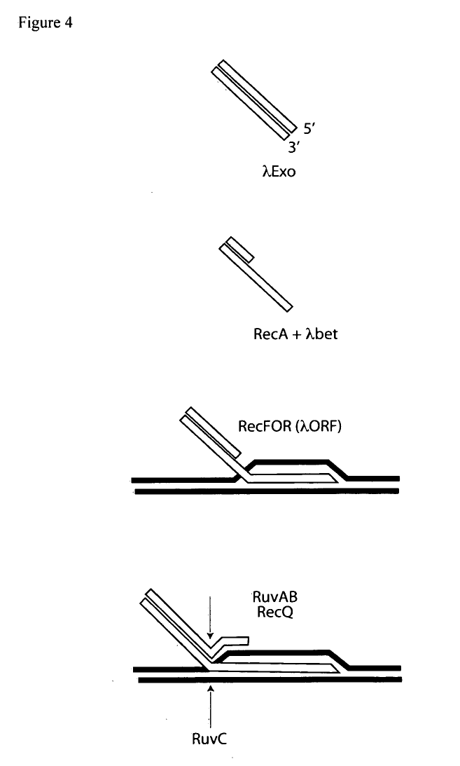

FIG. 4., summarizes the mechanisms of Red gam mediated gene replacement in E. coli

### DEFINITIONS

MSD: multiple strand displacement amplification

Red Gam: The exo, bet, and gam λ genes involved in Red recombination

Recombineering genes: Recombineering utilizes recipient cells or organisms which express phage-derived protein pairs, for example, RecE/RecT from the Rac prophage, or Redα/Redβ(exo, bet) from λ phage or other homologous protein pairs or fusions. Optionally, λ phage gam and orf60a genes are also included (e.g. pKD46).

The invention relates to methods for trait transfer between microbial organisms.

The invention is a cost effective efficient method for transferring traits into a microbial host.

Strain engineering by Genome Mass Transfer (GMT) is a new methodology that facilitates the transfer of desirable traits freely between strains. The strategy is to directly transfer gDNA from a strain with a desired property into a recipient strain that expresses recombineering genes. Then the strain is selected for acquisition of the desired trait (FIG. 1). This methodology is faster, simpler, more adaptable to high throughput applications, and more efficient than conventional methodologies for trait transfer such as P1 transduction.

### TRAIT TRANSFER PREFERRED EMBODIMENTS

In one preferred embodiment for trait transfer, genomic DNA is introduced into the host strain modified to express recombineering genes, whereupon the DNA becomes integrated into the genome through homologous recombination. In one preferred embodiment for trait transfer, genomic DNA is introduced into the host strain via electroporation. In yet another preferred embodiment for trait transfer, amplified DNA is introduced into the host strain via electroporation, whereupon the DNA becomes integrated into the genome by homologous recombination (FIG. 1). In yet another preferred embodiment for trait transfer, the amplified DNA is obtained directly from cell lysates, without prior purification. These ‘genome mass transfer’ processes dramatically decrease the time and complexity of trait transfer, while maintaining or improving host cell integrity (i.e. precision of gene transfer), relative to the processes described in the art.

Methods for isolation of genomic DNA from a variety of microbial species are known in the art, and included herein by reference (Ausubel F M. 1998. Current Protocols in Molecular Biology. Wiley Interscience).

Methods for amplification of DNA from cell lysates or purified gDNA are known in the art and included herein by reference (Abulencia C B, Wyborski D L, et al, 2006. Appl Environ. Microbiol. 72: 3291; Abulencia C, Keller M. US Patent application 2006/94033).

Methods for electroporation of large DNA molecules into microbial cells (such as yeast, or E. coli) are known in the art and included herein by reference (Sheng Y, Mancino V, Birren B. 1995. Nuc. Acids. Res. 23: 1990).

Methods for electroporation of genomic DNA molecules into microbial cells (such as E. coli, yeast or agrobacterium) are known in the art and included herein by reference (Choi et al, Supra, 2006; Charles et al, Supra 1994; Adam et al, Supra, 1999,).

Methods for direct gene transfer of DNA molecules from donor cells to recipient cells are known in the art and included herein by reference (Kilbane I I, J J, Bielaga B A. 1991. Biotechniques 10: 354; Nalin R, Sandrine D, Bertolla F, Buret F, Auriol P, Vogel T, Simonet P. Methods of creating genetic diversity US Patent Application 2004/0180350)

The combinational use of recombineering hosts for transfer of chromosomal traits between microbial cells using donor genomic DNA is not taught in the art. This combination provides a novel and enabling solution to the limitations of current gene transfer methods (such as P1 transduction or electroporation of genomic DNA) to transfer traits between microbial cells. While Choi et al, Supra, 2006 teach the use of electroporation of genomic DNA to transfer traits between Pseudomonas strains, and Charles et al, Supra 1994 teach to use electroporation of genomic DNA to transfer traits between Agrobacterium strains, the transfer frequency is too low for general application of the method for high efficiency strain engineering. As well, the prior art teaches away from using electroporation of genomic DNA to transfer traits between strains of E. coli since P1 transduction is still exclusively utilized for transfer of traits between E. coli strains. Recombineering host strains have been utilized for a variety of strain manipulation applications, but in all cases these applications use PCR, plasmid or oligonucleotides as the donor DNA. Thus the inventor's observation of efficient transfer of traits between E. coli strains using a recombineering host and genomic DNA is novel, unexpected, and enabling. While the inventor has demonstrated trait transfer utilizing a recombineering host in E. coli, an investigator skilled in the art can use the method in any microbial cell for which recombineering plasmids are available. Methods for introducing and expressing recombineering proteins in a wide variety of eukaryotic and prokaryotic species are know, including but not limited to Shigella, Salmonella, Yersinia, enteropathogenic or enterohemorrhagic E. coli, Caenorhabditis briggsae and are included herein by reference (Sawitzke J A, Thomason L C, Costantino N, Bubunenko M, Datta S, Court D L. 2007. Methods Enzymol 421: 171-199). Therefore the invention can be practiced generally in a variety of microbial cells or organisms (e.g. Caenorhabditis briggsae), including, but not limited to E. coli.

Once conceived as disclosed herein, this previously unsuggested combination of elements teaches a number of new and unexpected applications that will be clear to an investigator skilled in the art. Direct transfer of genomic DNA between E. coli strains can be used to rapidly assemble new strains for academic or industrial use. For example, transposon or antibiotic resistance marked gene knockout collections (for example, the Escherichia coli K-12 gene knockout collection as disclosed in Baba T, Ara T, Hasegawa M, Takai Y, Okumura Y, Baba M, Datsenko K A, Tomita M, Wanner B L, Mori H.2006. Mol. Syst. Biol 2006.0008; the E. coli transposon collection as described in Gerdes S Y et al. 2003 J Bacteriol 185: 5673-5684)) can be utilized as source DNA to rapidly transfer one or more traits to any other desired E. coli strain. Using amplified DNA eliminates the need for initial DNA purification, affording high throughput application of the invention. This allows unprecedented simplicity and speed in assembling rationally designed gene knockout combinations in new strains.

In one preferred embodiment, electroporation is used to introduce donor DNA into recipient strains. In alternative preferred embodiments, alternative methods for introduction of the DNA into host cells, for example, liposomal-mediated methods as disclosed by Kawata Y, Yano S, Kojima H.2003. Biosci. Biotechnol. Biochem. 67: 1179) or carbon nanotubes as disclosed by Rojas-Chapana J Troszczynska J, Firkowska I, Moresczeck C, Giersig M. 2005. Lab Chip 5: 536) are utilized. In yet another alternative preferred embodiment, electroporation efficiency is enhanced using methods known in the art. For example, cationic compounds can be used to increase the efficiency of electroporation per se, or electroporation of large DNA or amplified large DNA. Such methods have been developed for enhancing DNA delivery to eukaryotic cells and are incorporated herein by reference (De Jong G, Vanderbyl S L, Oberle V, Hoekstra D. US Patent Application 2003/0059940). Alternatively, tRNA assisted precipitation may be used (Zhu H, Dean R A. 1999. Nuc. Acids. Res. 27: 910), or addition of sugar such as sorbitol (Greener A L, Jerpseth B D. 2003 U.S. Pat. No. 6,586,249) or cysteine (Maas, R, 2005 U.S. Pat. No. 6,849,455).

### EXAMPLES

The method of the invention is further illustrated in the following examples. These are provided by way of illustration and are not intended in any way to limit the scope of the invention.

### Example 1

### Genome Mass Transfer (GMT)

Recombineering in E. coli is often based on either the phage Red or the RecET recombination functions. The λ genes involved in Red recombination are exo, bet, and gam (herein referred to as “red gam”). The exo gene product has 5′ to 3′ exonuclease activity, and the bet gene product is a single-strand DNA binding protein that promotes annealing. The gam gene product inhibits the RecBCD nuclease preventing linear DNA (i.e. PCR product) degradation. The red+gam+pKD46 plasmid (FIG. 2) was originally developed for recombineering and contains arabinose inducible exo, bet, and gam and orf60a genes in a conditional (temperature sensitive) replication plasmid (maintained at 30° C., lost at 42° C.) (Datsenko K A, Wanner B L. 2000 Proc. Natl. Acad. Sci.; 97:6640-6645). Briefly, for PCR mediated deletion of genes, an antibiotic resistance gene is PCR amplified using primers containing sequences homologous to the integration site [usually 50 base pairs (bp) at each end]. Conditionally replicating (requires pir+ host strain) plasmids pKD3 (chloramphenicol) and pKD4 (kanamycin) are used as templates for making gene knockout PCR fragments. To insert genes, the gene of interest and flanking antibiotic resistance gene are used as templates. The target strain, DH5α for example, is transformed with the ampicillin resistant lambda Red+gam+ containing plasmid pKD46 and Red gam production induced with arabinose. The cells are prepared and electroporated with the PCR fragment. Homologous recombinants are selected with kanamycin and cured of the pKD46 helper plasmid by shifting to the non-permissive temperature (pKD46 has a temperature sensitive origin of replication) and loss of ampicillin resistance verified. This technique has been utilized successfully in recA- strains such as DH5α, a common plasmid production host but the integration frequency is improved in such strains by inclusion of the recA+ gene on the pKD46 plasmid. The plasmids (e.g. pKD3, pKD4, pKD46) and pir+replication hosts (e.g. BW23474 for pKD3 and pDK4 propagation) are generally available from the E. coli Genetic Resource Center (Dept. of Molecular, Cellular, and Developmental Biology Yale University). This basic system has been adapted for use in a variety of other organisms.

These recombineering systems have not been utilized to transfer traits between strains. The target sequence is approximately 200 fold less abundant in gDNA [The E. coli genome is approximately 4000 kb, so the target sequence will be represented in 0.5% (1:200) of 20 kb fragments] compared to a PCR product so the frequency of transfer would be expected, by one skilled in the art, to be up to 200 fold reduced compared to PCR mediated gene knockout. This would be too low for practical application.

Despite this limitation, we evaluated the applicability of lambda red gam to trait transfer between strains since a method for direct electroporation of gDNA from a donor to a recipient strain would be dramatically simpler and more efficient than conventional methodologies to transfer traits such as P1 transduction.

Transfer of a chromosomally integrated gene cassette encoding the chloramphenicol resistance marker-Arabinose inducible lambda endolysin from the JM109 derived Xja strain (Zymo Research) into both DH5α and DH10B cell lines was assessed as follows.

The DH5α and DH10B cell lines were made proficient for recombination by co transformation of both the lambda red+gam+ plasmid pKD46 (Datsenko and Wanner, Supra 2000) and the pACYCTet RecA+ plasmid. pACYCTetRecA+ contains the E. coli recA+ gene (expressed from its own promoter), along with tetracycline resistance, in the pACYC184 backbone. The two plasmids are compatible and selected after transformation with ampicillin (pKD46) and tetracycline. Transformed cells were grown to midlog (0.4 OD₆₀₀/mL) in LB media containing 100 ug/mL ampicillin and 0.2% arabinose (to induce recombineering genes) and made electrocompetent by centrifugation and washing 2× in 10% glycerol and resuspending the final pellet in 10% glycerol (1/100 original culture volume).

Genomic DNA was prepared from the Xja strain (E. coli JM109 recA1 supE44 endA1 hsdR17 (rₖ⁻, mₖ⁺) gyrA96 relA1 thi mcrA Δ(lac-proAB)ΔaraB::λR, cat F′[traD36 proAB⁺lacI^{q} lacZ Δ M15]) using standard methodology (Ausubel, Supra. 1998, 2.4.2 miniprep procedure). Genomic DNA was extracted from 3 mL saturated culture and resuspended in a final volume of 100 uL of 1/10× TE. Concentration was estimated at approximately 100 ng/uL. The genomic DNA used was either untreated (high molecular weight >12 kb) or sonicated (smear of DNA from 100 by to 4 kb).

The electrocompetent cells (50 uL) were electroporated with 5 uL of genomic DNA (approximately 500 ng DNA). The control reaction was electroporated with 5 uL of a control 1.4 kb PCR product. The results are summarized in Table 1.

Surprisingly, the chromosomally integrated chloramphenicol resistance gene was successfully transferred at a high frequency using GMT from the donor strain (Xja) to two recipient strains (DH5α and DH10B). These results also demonstrate that the methodology is not strain specific. The transfer was confirmed by galactose phenotyping of two of the chloramphenicol resistant (chlor R) DH5α strains, which confirmed that the engineered chlorR strain had the gal- phenotype of recipient, not the gal+ of the donor. The process requires functional RecA+ protein, since no colonies were obtained with the pKD46 plasmid alone. In 5 out of 5 cases, the recombinant included the flanking lambda R endolysin (i.e. demonstrate autolysis), demonstrating the surprising feasibility of the approach for transfer of >2 kb (size of chlor R gene and lambdaR gene integrated in Xja strain; Jia, X, Kostal J, Claypool J A. US patent application 2006/0040393).

TABLE 1
Strain engineering by Genome Mass Transfer

| Strain | Electroporated DNA | # colonies | % autolysis |
|---|---|---|---|
| DH5α +pKD46 + | Xja HMW Genomic | 0 | |
| DH5α +pKD46 + | Xja HMW Genomic | 200 | 5/5+ |
| pACYCTetRecA+ | | | |
| DH5α + pKD46 + | Xja Sonicated | 58 | |
| pACYCTetRecA+ | Genomic | | |
| DH5α + pKD46 + | Control | 0 | |
| pACYCTetRecA+ | | | |
| DH10B + pKD46 + | Xja HMW Genomic | 23 | |
| pACYCTetRecA+ | | | |
| DH10B + pKD46 + | Xja Sonicated | 2 | |
| pACYCTetRecA+ | Genomic | | |
| DH10B + pKD46 + | Control | 0 | |
| pACYCTetRecA+ | | | |

+2/2 tested colonies were gal-

The relative contributions of RecA and lambda Red Gam (pKD46) were determined in a subsequent GMT experiment, summarized in Table 2.

TABLE 2
RecA and lambda Red Gam requirements for GMT

| Strain | Electroporated DNA | # colonies | % autolysis |
|---|---|---|---|
| DH5α | Xja HMW Genomic | 0 | |
| DH5α +pKD46+ | Xja HMW Genomic | 0 | |
| DH5α +pKD46+ | Xja HMW Genomic | 47 | (6/6) |
| pACYCTetRecA+ | | | |
| DH5α + | Xja HMW Genomic | 6 | (6/6) |
| pACYCTetRecA+ | | | |
| MG1655 | Xja HMW Genomic | 0 | |
| MG1655 +pKD46+ | Xja HMW Genomic | 2 | (2/2) |
| MG1655 +pKD46+ | Xja HMW Genomic | 3 | (3/3) |
| pACYCTetRecA+ | | | |
| MG1655+ | Xja HMW Genomic | 0 | |
| pACYCTetRecA+ | | | |

In DH5α GMT requires RecA, and the frequency of transfer is strongly enhanced by a combination of RecA and lambda Red Gam proteins. Consistent with this, GMT occurred in MG1655 (a recA+ strain) only when Red Gam proteins were introduced (Table 2). This synergy between the components may explain why GMT has not been previously identified, since most studies in the prior art tested electroporation transfer without lambda Red Gam proteins. Presumably, this synergy would also be observed with recA and proteins homologous to Red, for example, recET.

### Example 2

### Creation of pKD46-recA plasmids for GMT

To simplify GMT, the recA+ gene was transferred to pKD46. Two versions were made, with either the E. coli RecA or Pseudomonas aeruginosa RecA (RecAp_{A}) proteins.

pKD46-RecA

pKD46 vector was digested with NcoI, filled with klenow and dNTP, digested with SpeI, and the red+gam+ vector backbone gel purified (5205, 1124 bp). The E. coli RecA gene (expressed from its own promoter) was excised from the pDF25 vector (recA+) using KpnI (chewed blunt with T4 DNA polymerase and dNTP's) and SpeI and the recA+ gene purified (5037, 2465, 367). The two fragments were ligated and transformed into DH10B electrocompetent cells and ampicillin colonies that exhibited UV resistance (recA+) were isolated and the pKD46-RecA plasmid confirmed by restriction digestion.

pKD46-RecApa Orientation 1 and 2

The Pseudomonas Aeruginosa RecA (RecA_{PA}) protein induces hyper recombination in E. coli, in the absence of SOS induction, and presence or absence of E. coli recA protein (Baitin D M, Bakhlanova I V, Kil Y V, Cox M M, Lanzov V A. 2006 J. Bacteriol. 188: 5812-5820). A pKD46 vector was engineered to express RecA_{PA}, using the E. coli recA promoter and leader. A fusion with the E. coli RecA leader was made, since recA promoter from Pseudomonas is not functional in E. coli. The vector was also engineered to contain extra restriction sites at the junctions, to allow further modification. The pKD46 vector was digested with NcoI, CIP treated, and the linear vector gel purified (6.3 kb). The E. coli

RecA leader was PCR amplified from the pDF25 (recA+) vector using primers that amplified the promoter and leader sequence. The 200 by fragment was digested with the type IIS restriction enzyme AarI (Fermentas) to generate 5′ NcoI and 3′ unique 4 by non palindromic sequence) and gel purified. AarI type IIS enzyme digestion creates compatible sticky ends in the flanking DNA for cloning as follows. The 5′ end of the primer contains 4-6 bases, then the AarI site, 4 bases, then the 4 by sticky end of the NcoI (or, in the 3′ primer, the unique 4 by non palindromic sequence). Cleavage of the PCR product with AarI (Fermentas) cleaves after +4 and +8 (bottom strand) to generate a 4 by sticky end. Methods for use of AarI in cloning are disclosed in Williams, J A 2006 WO2006078979 and are included herein by reference. The Pseudomonas aeruginosa RecA gene was PCR amplified from genomic DNA (ATCC 47085D) using primers that were complementary to the 3′ unique 4 by non palindromic sequence to ligate to the E. coli recA promoter fragment, and at the 3′ complementary to NcoI, but the site is lost upon ligation. In this manner, the orientation can be determined by which side has a regenerated NcoI site (from the E. coli recA promoter fragment). The primers also introduce NheI, XhoI and SphI unique sites for cloning. The 1.2 kb PCR product was digested with AarI and gel purified. A 3 fragment ligation was performed, transformed into DHSalpha and ampicillin resistant colonies screened for UV resistance. All plating was performed at 30° C. to prevent loss of the temperature sensitive (ts) plasmid. Clones in both orientations were isolated and confirmed by sequencing (FIG. 2).

### Example 3

### Strain Engineering by GMT of Amplified DNA

GMT was performed as described in Example 1, comparing the efficiency of random primed isothermal amplified DNA to isolated genomic DNA. As well, transfer was demonstrated with a second marker, in this case an integrated gene transfer plasmid, pAH144-C1857-tetR. The pAH144 plasmid was developed for targeted gene insertion into E. coli (Haldimann and Wanner, 2001 J. Bacteriol 183; 6384-6393) at the phage HK022 attachment site. The integrated plasmid is selectable with Spectinomycin/Streptomycin, and the transfer of the intact plasmid can be assessed by transfer of the heat inducible tetracycline cassette (under the control of the phage lambda pR pL promoter and the lambda C1867ts repressor). The entire plasmid is 5 kb, so fragments larger than 5 kb must be transferred by GMT to confer both Spectinomycin/Streptomycin and tetracycline resistance.

Genomic DNA was amplified isothermally by multiple strand displacement amplification (MSD) using the GenomiPhi™ DNA Amplification Kit and random primers as per the manufacturer's instructions (Amersham). The results are summarized in Table 3.

TABLE 3
GMT of pAH144-C1857-tetR+

| | GMT gDNA | GMT gDNA | GMT Amplified |
|---|---|---|---|
| | 2 hr | 1 hr 37° C., | DNA |
| | 37° C. ++ | ½ hr 42° C. ++ | 2 hr 37° C. ++ |
| Strain | (recomb/plate) | (recomb/plate) | (recomb/plate) |
| DH5α | 0 | 0 | 0 |
| DH5α + | 0 | 0 | 0 |
| pKD46 + | | | |
| pACYCRecA+ | | | |
| DH5α + | 4 (4/4) | 10 (10/10) | 2 (2/2) |
| pKD46RecA | | | |
| DH5α + | 1 (1/1) | 0 | 2 (0/2) |
| pKD46RecA + | | | |
| pACYCLigase | | | |
| DH5α + | 2 (2/2) | 0 | 0 |
| pKD46RecA + | | | |
| pACYCpolI | | | |

+ NTC-tR Select Spec/Strep, confirm transfer with TetR ( )
++ Outgrowth conditions

This demonstrates transfer with a second target gene, and that GMT is feasible with both genomic DNA and amplified DNA. As well, the pKD46RecA plasmid is superior to the combination of two plasmids (DH5α+pKD46+pACYCRecA+). This may be due to lambda red gam mediated instability of the pACYCRecA+ plasmid by creation of linear concatamers; these can serve as substrates that titrate recombination proteins, and the RecA+ protein may be lost. This mechanism may also explain why no improvement was observed when ligase (pACYCLigase) or DNA polymerase I (pACYCPoll) were overexpressed along with the pKD46RecA plasmid.

Using the pKD46RecA plasmid, multiple strains have been engineered in DH5α. Trait transfers are summarized below:
Gene mutations transferred
   XJa autolytic cassette (chlor R)
      Genomic DNA or phi29 amplified DNA
   MutS (mutS:301 TNS, kanR)
   ydeA (miniTet JS1910, tetR)
   Red (recJ284:Tn10, tetR
Integrated plasmids (5 kb targets)
   NTC-T5RNase (integrated 5 kb C1857-T5RNase-lambdaR DHFR plasmid)
   NTC-tR (integrated 5 kb C1857-SpecR-StrepR-tetR plasmid)
      Genomic DNA or phi29 amplified DNA

Collectively, these results demonstrate the general utility of the method. The method is not specific to a strain (functional in MG1655, DH5α, DH10B) a resistance marker (chloramphenicol, tetracycline, kanamycin, spectinomycin streptomycin, DHFR markers have been transferred) or type of gene (transposon, integrated plasmid, and integrated PCR products have been transferred).

### Example 4

### Genetic Requirements for GMT Versus PCR Mediated Gene Knockout

Given the requirement for recA+ for GMT, GMT may require endogenous homologous recombination pathways involving DNA strand invasion. Homologous recombination by DNA strand invasion is summarized in FIG. 3. By contrast, red gam recombination mediated gene replacement using short linear double stranded DNA fragments follows a different pathway as summarized in FIG. 4. In brief, red gam recombination involves the following steps. The gam protein inhibits recBCD exonuclease, preventing digestion of the linear PCR product. The recombination protein is encoded by the red protein while the exo protein creates recombinogenic single stranded ends. Phage lambda bet and exo are homologous to the E. coli recE and recT proteins and can substitute for each other in PCR mediated gene knockout. Genetic requirements are discussed in detail in Poteete A R, Fenton A C. 2000 J. Bacteriol. 182: 2336-2340 and are included herein by reference.

GMT was used to create DH5α strains carrying various mutations in several of these recombination genes, to test the effect of different gene mutations or conditions on gene transfer efficiency. For example, mutS mutations and aminopurine addition inhibit mismatch repair, which increases the activity of recA mediated recombination. Methyl methane sulfonate (MMS) induces the SOS response which induces a variety of DNA recombination and repair enzymes. Heat shock induces many proteins, and has been shown to increase the frequency of PCR mediated gene replacement. DNA polymerase I (poll) and DNA ligase (lig) are involved in short strand DNA synthesis and ligating recombination products, respectively. Mutation in RecJ has been reported to enhance the frequency of PCR mediated gene replacement.

Using these strains containing pKD46recA or pKD46recApa orientation 2 plasmids as acceptor hosts, GMT was performed as described in examples 1 and 3. The efficiency of transfer of the Xja chloramphenicol resistance marker was determined. As well, the frequency of the standard PCR mediated gene replacement technique was determined, by targeting a chloramphenicol resistant PCR fragment to a nonessential site upstream of the mioC gene (using 50 by of mioC homology on the PCR fragment ends for targeting). 25 uL of cells were electroporated with gDNA or PCR fragment (approximately 200 ng), outgrown 2 hrs at 37° C. in SOC, and aliquots plated on LB+ chloramphenicol (6 ug/mL). A dilution of the cells was also plated on LB to determine total cell counts. PCR with primers that amplify only correct DNA insert junctions (combined insert internal and flanking primers) was utilized to determine the frequency of specific gene transfer or replacement (i.e. replacement precisely at the targeted site) versus non specific gene transfer (i.e. antibiotic resistance due to insertion of transferred cassette elsewhere in genome). The results are summarized in Table 4. Surprisingly, despite approximately 200 fold less target DNA in the gDNA [The E. coli genome is approximately 4000 kb, so the target sequence will be represented in 0.5% (1:200) of 20 kb fragments] the frequency of recovery of recombinants by GMT is actually elevated relative to PCR mediated gene replacement. High frequency GMT was observed with either the pKD46recApa or pKD46recA plasmids. The frequency of precise targeted replacement is also much higher; 47/48 (98%) for GMT versus 12/17 (71%) for PCR mediated gene replacement. This may be due to longer regions of homology increasing homologous versus non specific recombination. Overall, the optimal condition for GMT (pKD46RecA and aminopurine and MMS during culture growth) is greater than 1 log higher recombination frequency than the optimal condition for PCR mediated gene replacement (pKD46RecA and heat shock during culture growth). Despite using the pKD46 lambda Red+gam+ function, the genetic requirements for GMT are clearly distinct from those required for PCR mediated gene replacement (different genetic backgrounds improve GMT relative to PCR mediated gene replacement; Table 4), indicating that the as yet unknown mechanism driving highly efficient GMT is novel and not identical to known lambda red mediated gene replacement pathways. This may account for the dramatically higher frequency of GMT than would be expected based on sequence abundance. As well, based on these results, only a subset of the red, gam and exo genes may be necessary for GMT. The necessary subset of these genes can be determined by one skilled in the art, by individually deleting gam beta, exo and orf60a genes from pKD46 (FIG. 2) and determining the effect on GMT.

TABLE 4
GMT (Xja araB insert) versus PCR mediated gene knockout (mioC)

| | | | PCR | PCR | GMT | PCR |
|---|---|---|---|---|---|---|
| DH5α + pKD46RecA | GMT | GMT | knockout | knockout | (recomb/ | (recomb/ |
| (DRG) Strain modification | (recomb/mL) | specificity+ | (recomb/mL) | Specificity ++ | 10⁸ cell) | 10⁸ cell) |
| DRG +++ | 300 | 4/4 | 145 | 2/3 | 49 | 24 |
| DRG + Aminopurine (AP) | 965 | 4/4 | 0 | | 160 | 0 |
| DRG AP + MMS | 1270 | 4/4 | 35 | | 450 | 13 |
| DRG + MMS | 240 | 4/4 | 15 | 2/2 | 39 | 2.5 |
| DRG +++ | 265 | 4/4 | 85 | 3/4 | 49 | 16 |
| DRG + Heat shock | 85 | 4/4 | 210 | 3/4 | 11 | 27 |
| DRG +++ | 85 | 4/4 | 0 | | 19 | 0 |
| DRG + MutS | 70 | 4/4 | 30 | | 29 | 13 |
| DRG +++ | 85 | 4/4 | 10 | 1/1 | 18 | 2 |
| DRG + recJ- | 95 | 4/4 | 0 | | 28 | 0 |
| DRG +++ | 145 | 3/4 | 5 | | 41 | 1 |
| DH5α + pKD46RecApa | 130 | 4/4 | 30 | 1/3 | 38 | 9 |

+Gene replacement at araB verified by PCR. 47/48 colonies specific gene replacement
++ Gene replacement at mioC verified by PCR 12/17 specific
+++ Results using five independent lots of DRG competent cells are shown

GMT versus PCR mediate gene knockout was repeated as described above, using kanamycin resistant transfer markers: MutS (mutS:301 TNS, kanR) for GMT and a standard pKD4 based PCR product (150 ng) that targets spoT using 50 by homology regions. Recombinant cells were selected on LB+ kanamycin (10 ug/mL). Again, GMT was much higher frequency (2 logs) than PCR mediated gene knockout.

TABLE 5
GMT (mutS) versus PCR mediated gene knockout (spoT)

| DH5α + | | PCR | GMT | PCR |
|---|---|---|---|---|
| pKD46RecA | | knockout | (recomb/ | (recomb/ |
| (DRG) Strain | GMT | (recomb/ | 10⁸ | 10⁸ |
| modification | (recomb/mL) | mL) | cell) | cell) |
| DRG + polI + lig | 1985 | 0 | 950 | 0 |
| DRG + polI + lig | 1165 | 20 | 350 | 6 |
| (Heat shock) | | | | |
| DRG | 1065 | 0 | 460 | 0 |
| DRG (heat shock) | 1150 | 5 | 300 | 1 |

The frequency of GMT of NTC-T5RNase (integrated 5 kb C1857-T5RNase-lambdaR DHFR plasmid) is much higher, compared to MutS (mutS:301 TNS, kanR) (Table 6).

TABLE 6
GMT variability between loci

| DH5α + | | | | GMT NTC- |
|---|---|---|---|---|
| pKD46RecA | | GMT NTC- | GMT MutS | T5RNase |
| (DRG) Strain | GMT mutS | T5RNase | (recomb/10⁸ | (recomb/ |
| modification | (recomb/mL) | (recomb/mL) | cell) | 10⁸ cell) |
| DRG + | 1985 | 29,100 | 950 | 6767+ |
| polI + lig | | | | |
| DRG + | 1165 | 22,800 | 350 | 4145 |
| polI + lig | | | | |
| (Heat shock) | | | | |
| DRG | 1065 | 16,900 | 460 | 4023 |
| DRG | 1150 | 10,700 | 300 | 1672 |
| (heat shock) | | | | |

+1 in 6.6 × 10⁻⁵ cells is recombinant. If 20 kb DNA transferred, NTC-T5RNase represents 1/200 of total DNA.
Estimated total recombinant cells at 1.35% total viable cells

Collectively, these results demonstrate the general utility of the GMT strain engineering processes of the invention to improve strain development.

Thus, the reader will see that the compositions and production processes of the invention provide methods for improved strain engineering.

While the above description contains many specificities, these should not be construed as limitations on the invention, but rather as an exemplification of one preferred embodiment thereof. Many other variations are possible. For example, cells, zygotes and embryos of eukaryotes and other non-microbial species may be used. For example, Red/ET

Recombineering utilizes E. coli which express phage-derived protein pairs, either RecE/RecT from the Rac prophage, or Redα/Redβ (exo, bet) from λ phage. These protein pairs are functionally and operationally equivalent. RecE and Redα are 5″->3″ exonucleases, and RecT and Redβ are DNA annealing proteins. As well recombineering systems for Mycobacterium tuberculosis have been developed using mycobacteriophages (Che9c) encoded homologs of both RecE and RecT (Che9c gp60 and gp61 encode exonuclease and DNA-binding activities) (Van Kessel J C, Hatfull G F. 2007 Nat. Methods 4: 147-152).

Thus, RecET, mycobacteriophages, or other organisms recombineering gene functions could be substituted for lambda Redα/Redβ functions of the pKD46 plasmid for use in recombineering Mycobacterium or other eukaryotic or prokaryotic organisms. Accordingly, the scope of the invention should be determined not by the embodiments illustrated, but by the appended claims and their legal equivalents.

I claim:

1. A method for engineering microbial cells comprising the steps of:
a. isolating genomic DNA from a donor strain; and
b. introduction of donor DNA into a recipient strain engineered to express recombineering genes; and
c. selecting for acquisition of a trait in the recipient host;
whereby said method increases the frequency of targeted incorporation of said donor DNA into said recipient host's genome.

2) The method of claim 1 wherein said microbial cells are E. coli.

3) The method of claim 1 wherein said recombineering genes are lambda red, gam and/or exo or recET.

4) The method of claim 1 wherein said recombineering genes are transiently expressed from a conditional replication origin containing plasmid.

5) The method of claim 1 wherein the said genomic DNA from a donor strain is introduced into said recipient strain by electroporation.

6) The method of claim 1 wherein said step of isolating genomic DNA from a donor strain is performed by amplification of DNA from a donor cell lysate.

7) The method of claim 1 wherein said trait is an antibiotic resistant transposon insert that increases or decreases expression of a gene of interest.

8) A method for engineering cells comprising the steps of:
a. isolating genomic DNA from a donor strain; and
b. introduction of donor DNA into a recipient strain of the same or different species, which said recipient strain is engineered to express recombineering genes; and
c. selecting for acquisition of a trait in the recipient host;
whereby said method increases the frequency of targeted incorporation of said donor DNA into said recipient host's genome.

9) The method of claim 8, wherein said cells may include prokaryotic and/or eukaryotic cells, including zygotes, embryos, tissues and organisms.

10) The method of claim 8 wherein said recombineering genes are expressed by at least one means selected from the group consisting of: transient expression; from co-transfected genes or plasmids; from a conditional replication origin containing plasmid; or from integrated nucleic acid sequences.

11) The method of claim 8 wherein the said genomic DNA from a donor strain is introduced into said recipient strain by means of at least one mechanism selected from the following group: electroporation; transfection; liposomes; cationic lipids or polymers; calcium phosphate; carbon nanorods; or gene gun.

12) The method of claim 8 wherein said step of isolating genomic DNA from a donor strain is performed by amplification of DNA from a donor cell lysate.
